(19) United States
(12) Patent Application Publication
(10) Pub. No.: US 2024/0358376 A1
(43) Pub. Date: Oct. 31, 2024

(54) SYSTEMS AND METHODS FOR OCCLUDING VASCULAR DEFECTS

(71) Applicant: Covidien LP, Mansfield, MA (US)

(72) Inventor: Michael Khenansho, Irvine, CA (US)

(21) Appl. No.: 18/633,168

(22) Filed: Apr. 11, 2024

Related U.S. Application Data
(60) Provisional application No. 63/498,582, filed on Apr. 27, 2023.

(51) Int. Cl. A61B 17/12

(52) U.S. Cl. CPC A61B 17/12154; A61B 17/12113; A61B 2017/1205

(57) ABSTRACT

Systems and methods for occluding vascular defects are disclose herein. According to some embodiments, the present technology includes a method comprising releasing an occlusive member into an aneurysm cavity, the occlusive member comprising a mesh structure. Releasing the occlusive member can allow the occlusive member to self-expand into an expanded state in which the mesh structure defines an internal cavity. The occlusive device can be sized such that, when the occlusive device is positioned in the aneurysm cavity, a height of the occlusive device is less than a height of the aneurysm such that a space exists between a distal surface of the occlusive member and an inner surface of a dome of the aneurysm. The method may further comprise delivering a coil into the internal cavity of the mesh structure and not into the space between the occlusive member and the dome of the aneurysm.

### CROSS-REFERENCE TO RELATED APPLICATIONS

This application claims the benefit of and priority to U.S. Provisional Patent Application Ser. No. 63/498,582 filed Apr. 27, 2023, the entire disclosure of which is incorporated by reference herein.

### TECHNICAL FIELD

The present technology generally relates to systems and methods for occluding vascular defects.

### BACKGROUND

An intracranial aneurysm is a portion of an intracranial blood vessel that bulges outward from the blood vessel's main channel. This condition often occurs at a portion of a blood vessel that is abnormally weak because of a congenital anomaly, trauma, high blood pressure, or for another reason. Once an intracranial aneurysm forms, there is a significant risk that the aneurysm will eventually rupture and cause a medical emergency with a high risk of mortality due to hemorrhaging. When an unruptured intracranial aneurysm is detected or when a patient survives an initial rupture of an intracranial aneurysm, vascular surgery is often indicated. One conventional type of vascular surgery for treating an intracranial aneurysm includes using a microcatheter to dispose a platinum coil within an interior volume of the aneurysm. Over time, the presence of the coil should induce formation of a thrombus. Ideally, the aneurysm's neck closes at the site of the thrombus and is replaced with new endothelial tissue. Blood then bypasses the aneurysm, thereby reducing the risk of aneurysm rupture (or re-rupture) and associated hemorrhaging. Unfortunately, long-term recanalization (i.e., restoration of blood flow to the interior volume of the aneurysm) after this type of vascular surgery occurs in a number of cases, especially for intracranial aneurysms with relatively wide necks and/or relatively large interior volumes.

Another conventional type of vascular surgery for treating an intracranial aneurysm includes deploying a flow diverter within the associated intracranial blood vessel. The flow diverter is often a mesh tube that causes blood to preferentially flow along a main channel of the blood vessel while blood within the aneurysm stagnates. The stagnant blood within the aneurysm should eventually form a thrombus that leads to closure of the aneurysm's neck and to growth of new endothelial tissue, as with the platinum coil treatment. One significant drawback of flow diverters is that it may take weeks or months to form aneurysmal thrombus and significantly longer for the aneurysm neck to be covered with endothelial cells for full effect. This delay may be unacceptable when risk of aneurysm rupture (or re-rupture) is high. Moreover, flow diverters typically require antiplatelet therapy to prevent a thrombus from forming within the main channel of the blood vessel at the site of the flow diverter. Antiplatelet therapy may be contraindicated shortly after an initial aneurysm rupture has occurred because risk of re-rupture at this time is high and antiplatelet therapy tends to exacerbate intracranial hemorrhaging if re-rupture occurs. For these and other reasons, there is a need for innovation in the treatment of intracranial aneurysms. Given the severity of this condition, innovation in this field has immediate lifesaving potential.

### SUMMARY

The present technology generally relates to systems and methods for occluding vascular defects. The subject technology is illustrated, for example, according to various aspects described below, including with reference to FIGS. 1A-2C. Various examples of aspects of the subject technology are described as numbered clauses (1, 2, 3, etc.) for convenience. These are provided as examples and do not limit the subject technology.

Example 1. A method for treating an aneurysm, the method comprising: positioning a distal end of an elongate shaft in an aneurysm cavity; releasing an occlusive member from the elongate shaft into the aneurysm cavity, the occlusive member comprising a mesh structure, wherein releasing the occlusive member allows the occlusive member to self-expand into an expanded state in which the mesh structure defines an internal cavity, and wherein the occlusive device is sized such that, when the occlusive device is positioned in the aneurysm cavity, a height of the occlusive device is less than a height of the aneurysm such that a space exists between a distal surface of the occlusive member and an inner surface of a dome of the aneurysm; and delivering a coil into the internal cavity of the mesh structure and not into the space between the occlusive member and the dome of the aneurysm.

Example 2. The method of Example 1, wherein a combined volume of the mesh structure and the coil comprise no more than half of a volume of the aneurysm.

Example 3. The method of Example 1 or Example 2, wherein the height of the occlusive device is no more than half of a height of the aneurysm.

Example 4. The method of any one of the previous Examples, wherein the coil is detachably coupled to an elongate member, and wherein the coil and elongate member are configured to be advanced through a lumen of the elongate shaft.

Example 5. The method of any one of the previous Examples, wherein the elongate shaft is a first elongate shaft, and wherein a proximal portion of the occlusive member is detachably coupled to a distal end of a second elongate shaft configured to be advanced within a lumen of the first elongate shaft.

Example 6. The method of Example 5, wherein the coil is detachably coupled to an elongate member configured to be advanced through a lumen of the second elongate shaft.

Example 7. The method of any one of the previous Examples, wherein the mesh structure comprises a wall having a proximal portion, a distal portion, and a side portion extending therebetween, and wherein the mesh structure is positioned in the aneurysm such that the proximal portion of the mesh structure is positioned over the neck of the aneurysm.

Example 8. The method of any one of the previous Examples, wherein the mesh structure comprises a wall having a proximal portion, a distal portion, and a side portion extending therebetween, and wherein the mesh structure is positioned in the aneurysm such that the side portion of the mesh structure is in contact with an inner surface of a wall of the aneurysm.

Example 9. The method of any one of the previous Examples, wherein the mesh structure comprises a wall having a proximal portion, a distal portion, and a side portion extending therebetween, and wherein the mesh structure is positioned in the aneurysm such that the distal portion of the mesh structure is between the proximal portion and a dome of the aneurysm, and wherein the distal portion is spaced apart from the dome of the aneurysm.

Example 10. The method of any one of the previous Examples, wherein the coil is a first coil and the method further comprises delivering a second coil to the internal cavity of the mesh structure.

Example 11. A method for treating an aneurysm, the method comprising: positioning a mesh structure in an expanded state within an aneurysm cavity such that a proximal portion of the mesh structure is positioned over the neck of the aneurysm, a distal portion of the mesh is between the proximal portion and a dome of the aneurysm, spaced apart from the dome, and a side portion of the mesh structure, defined as a region of the mesh between the proximal and distal portions, is in contact with an inner surface of the aneurysm wall, wherein the mesh structure defines an internal cavity in the expanded state; and delivering a coil into the internal cavity of the mesh structure and not into a space between the distal portion of the mesh structure and the dome of the aneurysm.

Example 12. The method of Example 11, wherein a combined volume of the mesh structure and the coil comprise no more than half of a volume of the aneurysm.

Example 13. The method of Example 11 or Example 12, wherein a height of the mesh structure is no more than half of a height of the aneurysm.

Example 14. The method of any one of Examples 11 to 13, further comprising advancing the mesh structure in a low-profile, constrained configuration through a lumen of an elongate shaft.

Example 15. The method of Example 14, wherein the coil is detachably coupled to an elongate member, and wherein the coil and elongate member are configured to be advanced through a lumen of the elongate shaft.

Example 18. The method of Example 15, wherein the elongate shaft is a first elongate shaft and the mesh structure is coupled to a distal end of a second elongate shaft, and wherein the elongate member and coil are configured to be advanced through a lumen of the second elongate shaft.

Example 19. The method of Example 16, further comprising: detaching the coil from the elongate member to leave the coil within the internal cavity of the mesh structure, removing the elongate member from the lumen of the second elongate shaft, detaching the mesh structure from the second elongate shaft, and removing the first and second elongate shafts from the vasculature.

Example 20. The method of any one of Examples 11 to 16, wherein the coil is a first coil and the method further comprises delivering a second coil to the internal cavity of the mesh structure.

Example 21. The method of any one of Examples 11 to 17, wherein the mesh structure is globular in the expanded state.

Example 22. The method of any one of Examples 11 to 17, wherein the distal portion of the mesh structure comprises a substantially flat distal portion in the expanded state.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a partially schematic view of a system for treating an aneurysm in accordance with embodiments of the present technology.

FIG. 1B is an enlarged cross-sectional view of a distal portion of the delivery system shown in FIG. 1A.

FIGS. 2A-2C show an example method of treating an aneurysm with the systems of the present technology. The present technology relates to systems, methods, and devices for treating vascular defects such as aneurysms. In some embodiments, the methods described herein include delivering one or more coils into the internal cavity of a mesh structure positioned within the aneurysm sac. Together the coils and mesh structure are configured to fill only a portion of the internal volume of the aneurysm, which advantageously leads to faster healing as there is less volume and/or surface area for the aneurysm to heal around (as compared to conventional occlusive devices that fill the majority or all of the volume of the aneurysm). Moreover, using coils to fill the mesh structure, rather than a liquid embolic, avoids the many challenges associated with liquid embolic delivery, such as loss of the liquid embolic through the pores of the mesh into the parent vessel due to delayed congealing of the liquid composition within the aneurysm.

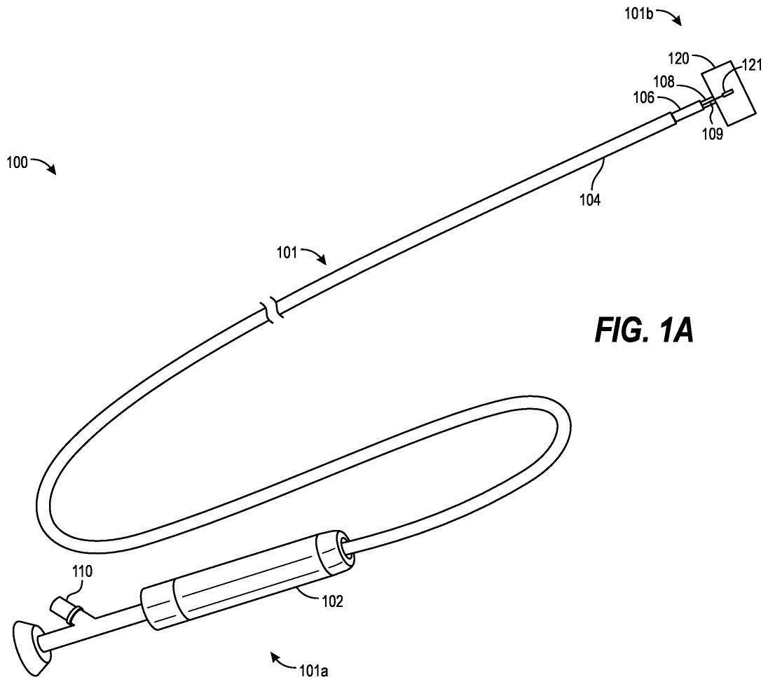

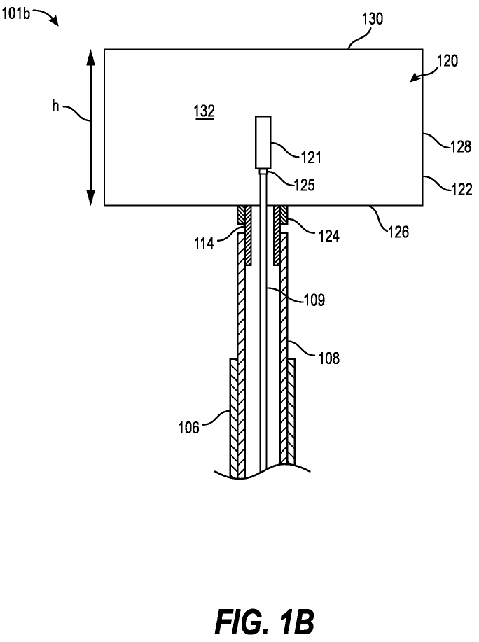

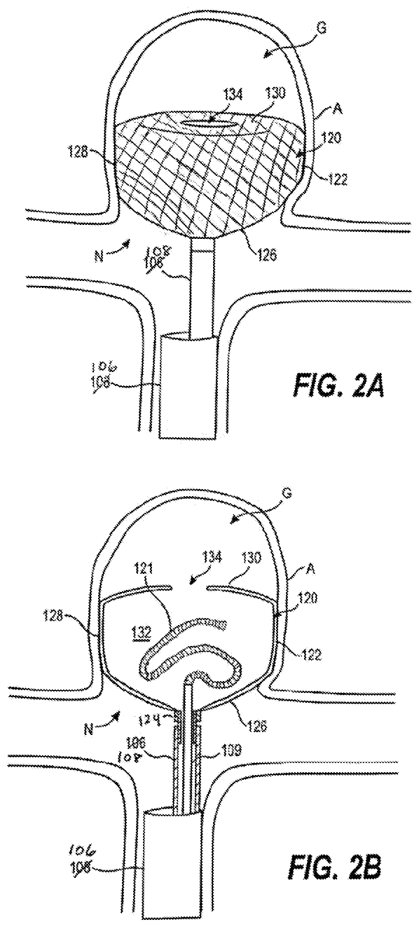

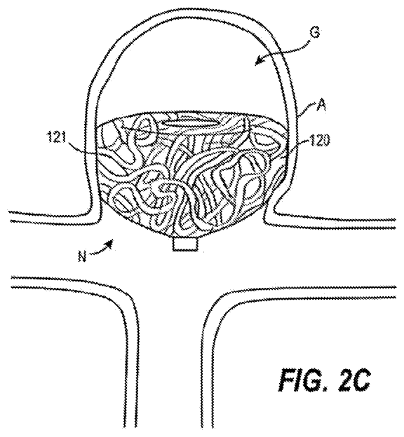

Specific details of devices, systems, and methods for treating aneurysms and/or other vascular defects in accordance with embodiments of the present technology are described herein with reference to FIGS. 1A-2D. FIG. 1A shows a system 100 for treating aneurysms, such as cerebral aneurysms, according to one or more embodiments of the present technology. As shown in FIG. 1A, the system 100 comprises a delivery system 101, a mesh structure 120 (shown schematically), and one or more coils 121 (shown schematically). The mesh structure 120 and coils 121 are configured to be detachably coupled to different components of the delivery system 101. The delivery system 101 is configured to intravascularly position the mesh structure 120 within an aneurysm, across the neck of the aneurysm, and further deliver the coils 121 into an interior cavity of the mesh structure 120. As detailed below, the mesh structure 120 prevents migration of the coils 121 into the parent vessel, and together the mesh structure 120 and coils 121 prevent blood from flowing into the aneurysm.

As shown in FIG. 1A, the delivery system 101 has a proximal portion 101a configured to be extracorporeally positioned during treatment and a distal portion 101b configured to be intravascularly positioned at or within an aneurysm. The delivery system 101 may include a handle 102 at the proximal portion 101a and a plurality of elongate shafts extending between the handle 102 and the distal portion 101b. In some embodiments, for example as shown in FIG. 1A, the delivery system 101 may include a first elongate shaft 104 (such as a guide catheter or balloon guide catheter), a second elongate shaft 106 (such as a microcatheter) configured to be slidably disposed within a lumen of the first elongate shaft 104, a third elongate shaft 108 configured to be slidably disposed within a lumen of the second elongate shaft 106, and an elongate member 109 configured to be slidably disposed within the lumen of the third elongate shaft 108. The delivery system 101 and/or the third elongate shaft 108 is configured to be detachably coupled at its distal end portion to the mesh structure 120 via a connector 124 (see FIG. 1B) of the mesh structure 120. Likewise, the delivery system 101 and/or the elongate member 109 is configured to be detachably coupled at its distal end portion to the coils 121 via a connector 125 (see FIG. 1B). In some variations, the delivery system 101 includes a plurality of elongate members 109 and associated coils 121, as in certain cases multiple coils may be required to sufficiently fill the volume of the mesh structure 120. In some embodiments, the delivery system 101 does not include the first elongate shaft 104.

The second elongate shaft 106 is generally constructed to track over a conventional guidewire in the cervical anatomy and into the cerebral vessels associated with the brain. The second elongate shaft 106 may also be chosen according to several standard designs that are generally available. For example, the second elongate shaft 106 can have a length that is at least 125 cm long, and more particularly may be between about 125 cm and about 175 cm long. The lumen of the second elongate shaft 106 is configured to slidably receive the mesh structure 120 in a radially constrained state. The second elongate shaft 106 can have an inner diameter of about 0.015 inches (0.0381 cm), about 0.017 inches (0.043 cm), about 0.021 inches (0.053 cm), or about 0.027 inches (0.069 cm).

The third elongate shaft 108 can be movable within the first and/or second elongate shafts 104, 106 to position the mesh structure 120 at a desired location. The third elongate shaft 108 can be sufficiently flexible to enable manipulation, e.g., advancement and/or retraction, of the mesh structure 120 through tortuous passages. Tortuous passages can include, for example, catheter lumens, microcatheter lumens, blood vessels, urinary tracts, biliary tracts, and airways. The third elongate shaft 108 can be formed of any material and in any dimensions suitable for the task(s) for which the system 100 is to be employed. In some embodiments, at least the distal portion of the third elongate shaft 108 can comprise a flexible metal hypotube. The hypotube, for example, can be laser cut along all or a portion of its length to impart increased flexibility. In some embodiments, the third elongate shaft 108 can be surrounded over some or all of its length by a lubricious coating, such as polytetrafluoroethylene (PTFE).

In some embodiments, the delivery system 101 can comprise a detachment portion 114 (FIG. 1B) comprising a tube extending between a distal end of the third elongate shaft 108 and the connector 124 of the mesh structure 120. Once the coil(s) have sufficiently filled an internal region of the mesh structure 120, a portion of the tube can be electrolytically dissolved to sever a region of the detachment portion 114 exposed between the third elongate shaft 108 and the mesh structure 120. Other means for electrolytic and mechanical detachment means are possible.

The mesh structure 120 may comprise an expandable element having a low-profile or constrained state while positioned within a catheter (such as the second elongate shaft 106) for delivery to the aneurysm and an expanded, deployed state for positioning within the aneurysm. In some embodiments the mesh structure 120 comprises a wall 122 (shown schematically in FIG. 1B) and a connector 124 coupled to a proximal portion of the wall 122. The wall 122 can define an interior region 132 when the mesh structure 120 is in an expanded state. As previously mentioned, the connector 124 is configured to be coupled to one or more components of the delivery system 101, such as the third elongate shaft 108 and/or detachment portion 114. The wall 122 of the mesh structure 120 can be formed of a resilient material and shape set such that upon exiting the second elongate shaft 106, the mesh structure 120 self-expands to a predetermined shape. The mesh structure 120 can have any shape or size in the expanded state that enables the mesh structure 120 to cover the aneurysm neck. In some embodiments, for example as shown in FIG. 2A, the mesh structure 120 can be configured to assume a hemispherical shape. Other shapes are possible, such as a spherical shape, a barrel shape, a disc shape, etc.

As shown in FIG. 1B, the wall 122 of the mesh structure 120 can comprise a proximal portion 126, a side portion 128, and a distal portion 130. The proximal portion 126 can be the portion of the wall 122 closest to the connector 124 and configured to be positioned over the neck of the aneurysm when the mesh structure 120 is implanted within the aneurysm. The distal portion 130 of the wall 122 can be configured to be positioned between the proximal portion 126 and a dome of the aneurysm, spaced apart from the dome of the aneurysm. The side portion 128 of the wall 122 extends between the proximal and distal portions 126, 130 and is configured to be in contact with an inner surface of the aneurysm. In some variations, for example as shown in FIG. 2A, all or a portion of the distal portion 130 of the wall 122 can be relatively flat. In other embodiments, all of a portion of the distal portion 130 of the wall 122 can be rounded. According to certain embodiments, the distal portion 130 can define an opening extending therethrough (as shown in FIG. 2A), while in other embodiments the distal portion 130 does not include an opening (other than the pores of the mesh wall) such that the wall 122 is continuous across the top of the mesh structure 120.

Referring still to FIG. 1B, the mesh structure 120 can have a height h in the expanded, implanted state measured between the most distal point along the distal portion 130 and the most proximal point along the proximal portion 126. As discussed below with reference to FIGS. 2A-2D, the height h of the mesh structure 120 can be less than a height of the aneurysm such that, when the mesh structure 120 is implanted within the aneurysm, a gap remains within in the aneurysm between the distal portion 130 of the mesh structure 120 and the dome of the aneurysm. In some embodiments, the mesh structure 120 is sized such that a height h of the mesh structure 120 is no more than half the height of the targeted aneurysm, and in some cases no more than a quarter of the height of the aneurysm. Likewise, a volume of the mesh structure 120 (in the expanded state) may be no more than half the volume of the aneurysm, and in some cases no more than a quarter of the volume of the aneurysm.

In some embodiments, the wall 122 of the mesh structure 120 is formed of a plurality of braided filaments that have been heat-set to assume a predetermined shape when released from the constraints of the delivery catheter. The wall 122 may be formed of metal wires, polymer wires, or both, and the wires may have shape memory and/or superelastic properties. The wall 122 may be formed of 24, 32, 36, 48, 64, 72, 96, 128, or 144 filaments. The wall 122 may be formed of a range of filament or wire sizes, such as wires having a diameter of from about 0.0004 inches to about 0.0020 inches, or of from about 0.0009 inches to about 0.0012 inches. In some embodiments, each of the wires or filaments have a diameter of about 0.0004 inches, about 0.0005 inches, about 0.0006 inches, about 0.0007 inches, about 0.0008 inches, about 0.0009 inches, about 0.001 inches, about 0.0011 inches, about 0.0012 inches, about 0.0013 inches, about 0.0014 inches, about 0.0015 inches, about 0.0016 inches, about 0.0017 inches, about 0.0018 inches, about 0.0019 inches, or about 0.0020 inches. In some embodiments, all of the filaments of the braided mesh may have the same diameter. For example, in some embodiments, all of the filaments have a diameter of no more than 0.001 inches. In some embodiments, some of the filaments may have different cross-sectional diameters. For example, some of the filaments may have a slightly thicker diameter to impart additional strength to the braid. In some embodiments, some of the filaments can have a diameter of no more than 0.001 inches, and some of the filaments can have a diameter of greater than 0.001 inches. The thicker filaments may impart greater strength to the braid without significantly increasing the device delivery profile, with the thinner wires offering some strength while filling out the braid matrix density.

In some embodiments, the mesh structure 120 can be a non-braided structure, such as a laser-cut stent. Moreover, the wall 122 of the mesh structure 120 may comprise a single mesh layer, or multiple mesh layers.

According to several methods of use, a physician may begin by intravascularly advancing the second elongate shaft 106 towards an intracranial aneurysm with the mesh structure 120 in a low-profile, collapsed state and coupled to a distal end portion of the third elongate shaft 108. A distal portion of the second elongate shaft 106 may be advanced through a neck of the aneurysm to locate a distal opening of the second elongate shaft 106 within an interior cavity of the aneurysm. The third elongate shaft 108 may be advanced distally relative to the second elongate shaft 106 to push the mesh structure 120 through the opening at the distal end of the second elongate shaft 106, thereby releasing the mesh structure 120 from the shaft 108 and enabling the mesh structure 120 to self-expand into an expanded, deployed state.

FIG. 2A shows the mesh structure 120 in an expanded, deployed state, positioned in an aneurysm cavity and still coupled to the third elongate shaft 108. In the expanded, deployed state, the height h (see FIG. 1B) of the mesh structure 120 can be less than a height of the aneurysm such that a gap G remains within in the aneurysm A between the distal portion 130 of the mesh structure 120 and the dome of the aneurysm A.

FIG. 2B is a cross-sectional view of the mesh structure 120 still attached to the delivery system as the coil(s) 121 are being delivered. As illustrated in FIG. 2B, one or more coils 121 can be advanced (e.g., pushed via the elongate member 109) through the second elongate shaft 106, connector 124, and proximal portion 126 of the mesh structure 120 to the interior region 132 of the mesh structure 120. During and after delivery, the coil(s) 121 exert a substantially uniform downward pressure (i.e., towards the parent vessel) on the mesh structure 120 that further seals and stabilizes the mesh structure 120 around the neck N of the aneurysm A. In some embodiments, the coil(s) 121 completely or substantially completely occlude the pores of the adjacent layer or wall of the mesh structure 120 such that blood cannot flow past the coil(s) 121 into the aneurysm cavity. It may be desirable to occlude as much of the mesh structure 120 as possible, as leaving voids of gaps could enable blood to flow in and/or pool, which may continue to stretch out the walls of aneurysm A. Dilation of the aneurysm A can lead to recanalization and/or herniation of the mesh structure 120 and/or embolic composition 202 into the parent vessel and/or may cause the aneurysm A to rupture. In some embodiments, the coil(s) 121 may fill greater than 40%, 50%, 60%, 65%, 70%, 75%, 80%, 85%, or 90% of the volume of the interior region 132 of the mesh structure 120.

As shown in FIG. 2C, once delivery of the coil(s) 121 is complete, the delivery system 101 and/or third elongate shaft 108 can be detached from the mesh structure 120 (electrolytically or mechanically) and withdrawn from the patient's body. The mesh structure 120 is shown transparently in FIG. 2C for ease of viewing the coil(s) 121. The coil(s) 121 and mesh structure 120 together fill only a portion of the internal volume of the aneurysm A such that a gap G exists between the mesh structure 120 and a dome of the aneurysm. This less-than-complete filling advantageously leads to faster healing as there is less volume and/or surface area for the aneurysm to heal around (as compared to conventional occlusive devices that fill the majority or all of the volume of the aneurysm).

### CONCLUSION

Although certain embodiments of these devices, systems, and methods may be described herein primarily or entirely in the context of treating saccular intracranial aneurysms, other contexts are within the scope of the present technology. For example, suitable features of described systems, devices, and methods for treating saccular intracranial aneurysms can be implemented in the context of treating non-saccular intracranial aneurysms, abdominal aortic aneurysms, thoracic aortic aneurysms, renal artery aneurysms, arteriovenous malformations, tumors (e.g. via occlusion of vessel(s) feeding a tumor), perivascular leaks, varicose veins (e.g. via occlusion of one or more truncal veins such as the great saphenous vein), hemorrhoids, and sealing endoleaks adjacent to artificial heart valves, covered stents, and abdominal aortic aneurysm devices, among other example. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-2C.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method for treating an aneurysm, the method comprising:
positioning a distal end of an elongate shaft in an aneurysm cavity;
releasing an occlusive member from the elongate shaft into the aneurysm cavity, the occlusive member comprising a mesh structure, wherein releasing the occlusive member allows the occlusive member to self-expand into an expanded state in which the mesh structure defines an internal cavity, and wherein the occlusive device is sized such that, when the occlusive device is positioned in the aneurysm cavity, a height of the occlusive device is less than a height of the aneurysm such that a space exists between a distal surface of the occlusive member and an inner surface of a dome of the aneurysm; and
delivering a coil into the internal cavity of the mesh structure and not into the space between the occlusive member and the dome of the aneurysm.

2. The method of claim 1, wherein a combined volume of the mesh structure and the coil comprise no more than half of a volume of the aneurysm.

3. The method of claim 1, wherein the height of the occlusive device is no more than half of a height of the aneurysm.

4. The method of claim 1, wherein the coil is detachably coupled to an elongate member, and wherein the coil and elongate member are configured to be advanced through a lumen of the elongate shaft.

5. The method of claim 1, wherein the elongate shaft is a first elongate shaft, and wherein a proximal portion of the occlusive member is detachably coupled to a distal end of a second elongate shaft configured to be advanced within a lumen of the first elongate shaft.

6. The method of claim 5, wherein the coil is detachably coupled to an elongate member configured to be advanced through a lumen of the second elongate shaft.

7. The method of claim 1, wherein the mesh structure comprises a wall having a proximal portion, a distal portion, and a side portion extending therebetween, and wherein the mesh structure is positioned in the aneurysm such that the proximal portion of the mesh structure is positioned over the neck of the aneurysm.

8. The method of claim 1, wherein the mesh structure comprises a wall having a proximal portion, a distal portion, and a side portion extending therebetween, and wherein the mesh structure is positioned in the aneurysm such that the side portion of the mesh structure is in contact with an inner surface of a wall of the aneurysm.

9. The method of claim 1, wherein the mesh structure comprises a wall having a proximal portion, a distal portion, and a side portion extending therebetween, and wherein the mesh structure is positioned in the aneurysm such that the distal portion of the mesh structure is between the proximal portion and a dome of the aneurysm, and wherein the distal portion is spaced apart from the dome of the aneurysm.

10. The method of claim 1, wherein the coil is a first coil and the method further comprises delivering a second coil to the internal cavity of the mesh structure.

11. A method for treating an aneurysm, the method comprising:
positioning a mesh structure in an expanded state within an aneurysm cavity such that a proximal portion of the mesh structure is positioned over the neck of the aneurysm, a distal portion of the mesh is between the proximal portion and a dome of the aneurysm, spaced apart from the dome, and a side portion of the mesh structure, defined as a region of the mesh between the proximal and distal portions, is in contact with an inner surface of the aneurysm wall, wherein the mesh structure defines an internal cavity in the expanded state; and
delivering a coil into the internal cavity of the mesh structure and not into a space between the distal portion of the mesh structure and the dome of the aneurysm.

12. The method of claim 11, wherein a combined volume of the mesh structure and the coil comprise no more than half of a volume of the aneurysm.

13. The method of claim 11, wherein a height of the mesh structure is no more than half of a height of the aneurysm.

14. The method of claim 11, further comprising advancing the mesh structure in a low-profile, constrained configuration through a lumen of an elongate shaft.

15. The method of claim 14, wherein the coil is detachably coupled to an elongate member, and wherein the coil and elongate member are configured to be advanced through a lumen of the elongate shaft.

16. The method of claim 15, wherein the elongate shaft is a first elongate shaft and the mesh structure is coupled to a distal end of a second elongate shaft, and wherein the elongate member and coil are configured to be advanced through a lumen of the second elongate shaft.

17. The method of claim 16, further comprising:
detaching the coil from the elongate member to leave the coil within the internal cavity of the mesh structure,
removing the elongate member from the lumen of the second elongate shaft,
detaching the mesh structure from the second elongate shaft, and
removing the first and second elongate shafts from the vasculature.

18. The method of claim 11, wherein the coil is a first coil and the method further comprises delivering a second coil to the internal cavity of the mesh structure.

19. The method of claim 11, wherein the mesh structure is globular in the expanded state.

20. The method of claim 11, wherein the distal portion of the mesh structure comprises a substantially flat distal portion in the expanded state.
